(19) United States
(12) Patent Application Publication
(10) Pub. No.: US 2006/0094003 A1
(43) Pub. Date: May 4, 2006

(54) Acetylcholine-dependent current as a novel ionic target for atrial fibrillation

(76) Inventors: Stanley Nattel, Cote St-Luc (CA); Terence Hebert, Pointe-Claire (CA)

Correspondence Address: Louis Tessier, P.O. Box 54029, Town Mount-Royal, QC, H3P 3H4, CA

(21) Appl. No.: 11/068,161

(22) Filed: Mar. 1, 2005

Related U.S. Application Data
(60) Provisional application No. 60/623,243, filed on Nov. 1, 2004.

(51) Int. Cl. C12Q 1/00

(52) U.S. Cl. 435/4

(57) ABSTRACT

A method for treating a cardiac pathology. The method includes the administration to a mammal of a therapeutically effective amount of a substance interfering with an acetylcholine-dependent potassium current. Also, a method for identifying a compound for treating atrial fibrillation in a mammal.

This Application claims priority from U.S. Provisional Patent Application Ser. No. 60/623,243 filed Nov. 1, 2004.

### FIELD OF THE INVENTION

The present invention relates to the use of a constitutive acetylcholine-dependent current as a novel ionic target for atrial fibrillation therapy.

### BACKGROUND OF THE INVENTION

Atrial fibrillation (AF) is increasingly prevalent in the population due at least in part to obesity and to an aging demographics. Current treatments have many drawbacks, such as relatively large recurrence rates and more or less severe secondary effects.

Cardiac tissue in the pulmonary vein sleeves (PVs) are important for the initiation and maintenance of AF (Haissaguerre et al. 1998; Pappone et al. 2000). However, the cellular mechanisms underlying PV arrhythmogenicity are relatively obscure. Enhanced automaticity and triggered activity have been reported in isolated PV sleeve cardiomyocytes (Chen et al. 2001). PVs were found to show a time-dependent hyperpolarization-activated current that was increased by atrial tachycardia (AT), which was not characterized but was believed to represent a current generally known as If (Chen et al. 2001). In previous studies of PV ionic properties (for example Ehrlich et al. 2003), an hyperpolarization-activated inward currents was observed, but was found to be relatively sensitive to Ba²⁺, a pharmacological property inconsistent with the current I_{f} (DiFrancesco, 1993).

Against this background, there exists a need in the industry to provide a novel target for atrial fibrillation therapy.

Many publicly available documents are cited in this document, the contents of which are hereby incorporated by reference.

### OBJECTS OF THE INVENTION

An object of the present invention is therefore to provide a constitutive acetylcholine-dependent current as a novel ionic target for atrial fibrillation therapy.

### SUMMARY OF THE INVENTION

In a broad aspect, the invention provides a method for identifying a compound for treating atrial fibrillation in a mammal having a heart. The method includes:

a. subjecting the mammal to an atrial tachycardia remodeling treatment under conditions leading to a substantial increase in a constitutive Kir3 mediated acetylcholine-dependent potassium current in left atrial cardiomyocytes of the heart of the mammal;

b. isolating an atrial preparation from the heart of the mammal;

c. treating the atrial preparation with the compound;

d. submitting the atrial preparation to stimuli so as to attempt to produce tachyarrythmias; and

e. selecting the compound as a likely candidate for the treatment of atrial fibrillation if the compound substantially inhibits tachyarrythmias in the atrial preparation.

In another broad aspect, the invention provides a method for treating a cardiac pathology. The method includes the administration to a mammal of a therapeutically effective amount of a substance interfering with an acetylcholine-dependent potassium current.

In yet another broad aspect, the invention provides a method for treating atrial fibrillation. The method includes the administration to a mammal of a therapeutically effective amount of a substance inhibiting a constitutive acetylcholine-dependent potassium current.

In yet another broad aspect, the invention provides a constitutive acetylcholine-dependent potassium current embodied in a Kir3 ionic channel.

Advantageously, the method targets an ion channel which is important in the atrium, particularly in AF, and has not been observed in the ventricle. Therefore, drugs that target this channel have a potential to be effective in treating AF while reducing the risk of causing ventricular proarrhythmia.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 illustrates various I_{KH} current characteristics;

FIG. 2 illustrates effects of cation substitution and [K⁺]ₒ modulation on I_{KH};

FIG. 3 illustrates I_{KH} response to external Ba²⁺ and Cs⁺;

FIG. 4 illustrates neurohumoral modulation of I_{KH};

FIG. 5 illustrates the effect of tertiapin-Q on I_{KH} and action potentials;

FIG. 6 illustrates that elements of G protein signalling pathways affect I_{KH};

FIG. 7 illustrates atrial tachycardia-induced changes in I_{KH} and modulation by carbachol;

FIG. 8 illustrates immunofluorescence studies;

FIG. 9 illustrates immunoblots for Kir3 subunits, M₂ receptor and G_{αI};

FIG. 10 illustrates an effect of tertiapin on sustained atrial tachycardia in vitro; and

FIG. 11 illustrates an action potential duration change further to a treatment by tertiapin-Q

### DETAILED DESCRIPTION

A time-dependent potassium current has been characterized in canine cardiomyocytes. The current includes a hyperpolarization-activated time-dependent potassium current in canine cardiomyocytes from pulmonary vein myocardial sleeves and left atrium. Some properties of this new current, hereinafter I_{KH}, are described hereinbelow.

Cardiomyocytes from the pulmonary vein sleeves (PVs) are known to play a relatively important role in atrial fibrillation. PVs have been shown to exhibit time-dependent hyperpolarization-induced inward currents of uncertain nature. A time-dependent K⁺ current, hereinafter I_{KH}, was observed upon hyperpolarization of PV and left atrial (LA) cardiomyocytes (I_{KH}) and its biophysical and pharmacological properties were characterized.

The activation time constant was relatively weakly voltage dependent, ranging from 386±14 to 427±37 ms between −120 and −90 mV, and the half-activation voltage averaged −93±4 mV. I_{KH} was relatively larger in PV than LA cells (e.g. at −120 mV: −2.8±0.3 versus −1.9±0.2 pA pF⁻¹, respectively, P<0.01). The reversal potential was approximatively −84 mV with 5.4 mM[K⁺]ₒ and changed by 55.7±2.4 mV per decade [K⁺]ₒ change. I_{KH} was relatively Ba²⁺ sensitive, with a 50% inhibitory concentration (IC50) of 2.0±0.3 μM (versus 76.0±17.9 μM for instantaneous inward-rectifier current, P<0.01), and showed similar Cs⁺ sensitivity to instantaneous current.

I_{KH} was relatively potently blocked by tertiapin-Q, a 22-amino acid peptide synthesized from honeybee venom that is a selective Kir3-subunit channel blocker (IC50 10.0±2.1 nM), was unaffected by atropine and was increased by isoproterenol (isoprenaline), carbachol and the non-hydrolysable guanosine triphosphate analogue GTPS. I_{KH} activation by carbachol required GTP in the pipette and was prevented by pertussis toxin pretreatment.

Tertiapin-Q delayed repolarization in atropine-exposed multicellular atrial preparations studied with standard microelectrodes (action potential duration pre- versus post-tertiapin-Q: 190.4±4.3 versus 234.2±9.9 ms, PV; 202.6±2.6 versus 242.7±6.2 ms, LA; 2 Hz, P<0.05 each). Seven-day atrial tachypacing significantly increased I_{KH} (e.g. at −120 mV in PV: from −2.8±0.3 to 4.5±0.5 pA pF⁻¹, P<0.01).

In summary, I_{KH} is a time-dependent, hyperpolarization-activated K⁺ current that likely involves Kir3 subunits and appears to play a significant role in atrial physiology. I_{KH} has properties of a constitutively active acetylcholine-dependent current and is highly sensitive to tertiapin-Q (TQ, IC50 ˜10 nM), a relatively highly-selective Kir3 blocker.

The potential role of I_{KH} in atrial tachycardia (AT)-remodeled canine left atrial (LA) preparations with the use of tertiapin-Q (TQ) as a probe was assessed. A brief summary of this assessment is presented hereinbelow, followed by more details regarding the various experimental protocols that were used.

Briefly, dogs were subjected to 7-13 days of AT at about 400 beats per minute (bpm). LA preparations were then coronary-perfused and studied intact in vitro or subjected to cardiomyocyte isolation. I_{KH} was studied with patch clamp.

AT pacing increased I_{KH} at −110 mV from −2.2±0.6 pA/pF (control) to −3.8±0.7 pA/pF (AT) in LA cardiomyocytes, and the 100 nM TQ-sensitive component increased from −1.7±0.5 (control) to −2.8±0.5 (AT) pA/pF. Prolonged atrial tachyarrhythmias could be induced with single extrastimuli at varying cycle lengths in AT-remodeled, but not control preparations, but not controls. In AT-remodeled preparations, mean tachyarrhythmia duration was 11602±604 ms (n=81, 8 dogs), with a cycle length of 108±0.3 ms. Tachyarrhythmia duration was decreased statistically significantly by 100 nM TQ, to 520±6 ms (n=66, 8 dogs, P<0.05), and tachyarrhythmia cycle length increased to 179±0.8 ms (P<0.001). In 2 cases, tachyarrhythmia lasted uninterrupted for more than 20 minutes; TQ administration terminated arrhythmia within 4 minutes in both. Consistent with the antiarrhythmic actions observed, TQ prominently increased the duration of action potentials in AT-remodeled canine LA.

AT-remodeling increased I_{KH} and a highly-selective antagonist, TQ, increased action potential duration and suppressed atrial tachyarrhythmias in AT-remodeled preparations. Inhibition of the constitutive acetylcholine-related K+-current, as typified by but not excluded to I_{KH}, is a novel approach to the treatment of AF.

The above-summarized data suggests a method for treating a cardiac pathology comprising the administration to a mammal of a therapeutically effective amount of a substance interfering with an acetylcholine-dependent potassium-carried current.

For example, and non-limitatively, interfering with an acetylcholine-dependent potassium-carried current includes inhibiting a repolarization current so as to increase a duration of a repolarization phase in cardiomyocytes.

In a specific embodiment of the invention, the mammal is a human. In addition, a specific example of a cardiac pathology is atrial fibrillation. In some embodiments of the invention, the current is mediated by a Kir3 channel.

The above-summarized data also suggests a method for treating atrial fibrillation comprising the administration to a mammal of a therapeutically effective amount of a substance inhibiting a constitutive acetylcholine-related potassium current.

Also, a new a method for identifying a compound for treating atrial fibrillation in a mammal having a heart. The method includes:

a. subjecting the mammal to an atrial tachycardia remodeling treatment under conditions leading to a substantial increase in a constitutive Kir3 mediated acetylcholine-dependent potassium current in left atrial cardiomyocytes of the heart of the mammal;

b. isolating an atrial preparation from the heart of the mammal;

c. treating the atrial preparation with the compound;

d. submitting the atrial preparation to stimuli so as to attempt to produce tachyarrythmias; and

e. selecting the compound as a likely candidate for the treatment of atrial fibrillation if the compound substantially inhibits tachyarrythmias in the atrial preparation.

In addition, based on the evidence herein, other assays for efficacy against acetylcholine-dependent currents, such as current induced in cardiomyocytes or Kir3.1 and/or Kir3.4 expressing cell lines, are identified as screening methods for the identification of novel antiarrhythmic agents acting by the same or similar mechanisms.

Therefore, there is also suggested a method for identifying a compound for treating atrial fibrillation in a mammal. The method includes the steps of:

a. providing a cell into which a constitutive Kir3 mediated acetylcholine-dependent potassium current is present;

b. treating the cell with the compound; and

c. selecting the compound as a likely candidate for the treatment of atrial fibrillation if the compound substantially inhibits the constitutive Kir3 mediated acetylcholine-dependent potassium current.

The above-described methods increase a likelihood that the compound of clinical significance in the treatment of tachyarrythmia be identified. Therefore, a method for treating tachyarrythmias in a mammal includes administering the compound to the mammal. Examples of suitable mammals include dogs and humans, among others.

### EXAMPLE 1

Tissue and Cell Preparations

Adult mongrel dogs of either sex (weighing about 20-35 kg) were anaesthetized with pentobarbital (30 mg kg⁻¹ I.V.) and artificially ventilated with room air. Hearts and adjacent lung tissue were relatively quickly excised through a left lateral thoracotomy and immersed in oxygenated Tyrode solution (composition detailed hereinbelow) at room temperature. Removal of the heart and lungs produced circulatory arrest, resulting in relatively effective and relatively humane killing. A left atrial (LA) preparation with the PVs intact was perfused via the left circumflex coronary artery, and subjected to either standard fine-tipped microelectrode recording of action potentials (APs) or cardiomyocyte isolation with collagenase-containing solutions, as previously described (Ehrlich et al. 2003). Six dogs were subjected to atrial tachycardia-induced remodeling induced by 1 week of atrial pacing at about 400 beats min⁻¹ after ablation of the AV node, as described in Li et al. 1999. Animal care and handling procedures followed the guidelines of the Canadian Council on Animal Care.

To isolate LA and PV cardiomyocytes, the proximal circumflex artery was cannulated and the distal ends of PV myocardial sleeves (approximately 1-1.5 cm from the PV-LA junction) were marked with silk thread prior to subsequent enzyme perfusion with collagenase (100 U ml⁻¹, Worthington, type II), in order to facilitate localization of PV sleeves after enzymatic digestion. After a period of 45 min, epicardial tissue was removed and the underlying muscular sleeve of PVs was found to be relatively well digested, with the smooth muscle layer still intact and unaffected by the isolation procedure. With this method, PVs were relatively well-perfused and single cardiomyocytes could be isolated from all veins. Cardiomyocytes obtained from PVs were morphologically similar to LA cardiomyocytes isolated from the LA free wall in the same dogs. All comparisons were based on PV and LA cardiomyocytes isolated from each dog on each experimental day. After isolation, cells were stored at 4° C. and studied on the same day. For standard microelectrode experiments, intact tissue preparations including the LA and adjacent PVs were mounted in a chamber and perfused via the circumflex artery with oxygenated Krebs solution at 36±0.5° C. (see for example Kneller et al. 2002).

Electrophysiology

Currents were recorded with the whole-cell patch-clamp technique at 36±0.5° C., as described in Yue et al. 1996. All junction potentials were zeroed prior to formation of gigaohm seals. The compensated series resistance and capacitive time constant (τ) averaged 3.9±0.1 M and 257±81 μs, respectively, and voltage errors across the series resistance did not exceed 5 mV. Capacitance was assessed using 5 mV, 10 ms hyperpolarizing steps from a holding potential (HP) of −60 mV. Junction potentials averaged 11.8±0.9 mV and were not routinely corrected. Cell capacitance averaged 81±4 pF for PV and 69±8 pF for LA cardiomyocytes (n=83, 29 cells, respectively, P=n.s.). Atrial tachycardia did not affect cell capacitance (84±10 versus 91±10 pF, n=9 and 12 for PV and LA cells, respectively, P=n.s.). Original recordings are shown in terms of current amplitude, but mean data are presented as current density (pA pF−1) to control for variability in cell size.

Currents were recorded with hyperpolarizing and depolarizing pulses (generally 4 s duration) from a HP of −40 mV to selected test potentials (TPs). Recordings were repeated 3 times, and mean values obtained. For the determination of reversal potentials, tail currents were recorded after 1.6 s pulses to −120 mV followed by 3.2 s depolarizations to TPs between −110 and +20. All voltage protocols were delivered at 0.1 Hz.

Fine-tipped microelectrodes (resistance 15-20 M when filled with 3 M KCl) coupled to a high input-impedance amplifier were used to record APs as described in Kneller et al. 2002.

Solutions

Tyrode solution contained (mM): NaCl 136, KCl 5.4, MgCl₂ 1, CaCl₂ 1, NaH₂PO₄ 0.33, Hepes 5 and dextrose 10 (pH 7.35 with NaOH). The cell-storage solution contained (mM): KCl 20, KH₂PO₄ 10, dextrose 10, mannitol 40, L-glutamic acid 70, β-OH-butyric acid 10, taurine 20, EGTA 10 and 0.1% bovine serum albumin (pH 7.3, KOH). Nifedipine (5 μM) was used to suppress L-type Ca²⁺ current (I_{Ca}) in all experiments. 4-Aminopyridine (4-AP, 2 mM) was added to suppress transient outward current (Iₜₒ). Atropine was added as indicated to the extracellular solution to suppress muscarinic receptor-activated currents. Na+ current (INa) contamination was avoided by using a HP of −40 mV for recording of hyperpolarization-induced currents and by substitution of equimolar Tris-HCl for external NaCl for tail-current recordings. When different external K⁺ concentrations were applied, the osmolarity was kept constant by proportionate reduction of NaCl content in the solution. The standard internal solution contained (mM): potassium aspartate 110, KCl 20, MgCl2 1, MgATP 5, GTP (lithium salt) 0.1, Hepes 10, sodium phosphocreatine 5 and EGTA 5.0 (pH 7.3 with KOH). In experiments with K+-free internal solution, potassium aspartate was replaced by equimolar caesium aspartate and KCl by CsCl and pH was set to 7.3 with CsOH. For standard-microelectrode experiments, a solution containing (mM): NaCl 120, KCl 4, KH₂PO4 1.2, MgSO₄ 1.2, NaHCO₃ 25, CaCl₂ 1.25 and dextrose 5 (95% O₂-5% CO₂, pH 7.4) was used to perfuse the tissue.

Stock solutions of BaCl₂ (1 M) and CsCl (1 M) were produced initially and used throughout the experiments. Stock solution of isoproterenol was prepared under protection from light on the day of experiments and freshly prepared ascorbic acid (100 μM) was added in order to prevent isoproterenol oxidization. Carbachol (1 μM) was dissolved in Tyrode solution, tertiapin-Q in 0.1% acetic acid. GTPS (0.1 mM) was used in place of GTP in internal solutions for some experiments. For experiments involving pertussis toxin (PTX, stock solution dissolved in distilled H₂O) cells were incubated at 37° C. in 1.5 mg I-1 PTX for at least 9 h prior to experiments. Parallel controls were performed with cells from the same isolates incubated in the same fashion and the same solution, but without PTX. Vehicle alone did not affect the current. Unless otherwise specified, drugs were obtained from Sigma.

Western Blot, Immunofluorescence Studies and Confocal Imaging

After isolation of single cardiomyocytes, cells were suspended in lysis buffer (5 mM Tris pH 7.4, 2 mM EDTA, 5 mg ml⁻¹ trypsin inhibitor, 0.1 mg ml⁻¹ benzamidine, 0.43 mg ml⁻¹ leupeptin). After homogenization (2×10 s bursts with a Polytron homogenizer) and centrifugation (20 min, 16000 r.p.m), pellets were resuspended in a buffer (75 mM Tris, 12.5 mM EDTA, 2 mM MgCl₂). Proteins were fractionated on 7.5% SDS-PAGE gels, transferred to polyvinyl difluoride (PVDF) membranes and blotted with anti-Kir 3.1 (1:1000), anti-M2 receptor (1:500, both from Alomone), anti-Gi-3 (1:500, Santa Cruz) and anti-Kir 3.4 antibody (1 μg ml⁻¹). Bands were visualized with enhanced chemiluminescence. All immunoblot band intensity measurements were normalized to the GAPDH band intensity of the loaded sample (anti-GAPDH 1:5000, RDI).

LA and PV cardiomyocytes were seeded on glass coverslips (prepared with 15 μg ml⁻¹ laminin) for 1 h, fixed with 2% paraformaldehyde for 20 min, washed 3 times (5 min) with phosphate-buffered saline (PBS), then blocked with 2% normal donkey serum (Jackson Laboratories) and permeabilized with 0.2% Triton X-100 for 1 h in an incubation chamber. Cells were incubated with primary antibodies (anti-Kir 3.1 1:200, anti-Kir 3.2 1:400, both from Alomone), anti-Kir 3.4 1.3 μg ml⁻¹) overnight at 4° C., followed by three washes with PBS (5 min) and incubation with anti-rabbit secondary antibody (conjugated with tetra-methyl-rhodamine-isothiocyanate (TRITC)) for 1 h at room temperature, Molecular Probes). Cells were examined on an inverted laser-scanning microscope (LCM 510, Zeiss, Germany). TRITC was excited at 543 nm with a He—Ne laser and emitted fluorescence signals at 566 nm (red).

Specificity of primary antibodies for Kir 3.1, 3.2 and 3.4 was validated by immunofluorescent studies of transfected and non-transfected mammalian cells (Chinese hamster ovary cells; American Type Culture Collection, Manassas, Va.). Transfected cells showed clear staining, whereas no staining of non-transfected cells was observed with any of the primary antibodies (not shown).

Data Analysis

Clampfit 6.0 (Axon) and Graph Pad Prism 3.0 software were used for data analysis and non-linear curve fitting. Bands from immunoblots were analysed using QuantityOne software and immunofluorescence data were analysed using LSM Image Browser. Data are presented as means±S.E.M. and statistical comparisons were performed with Student's t test. P<0.05 was considered to indicate statistical significance.

Results

Voltage and Time Dependence

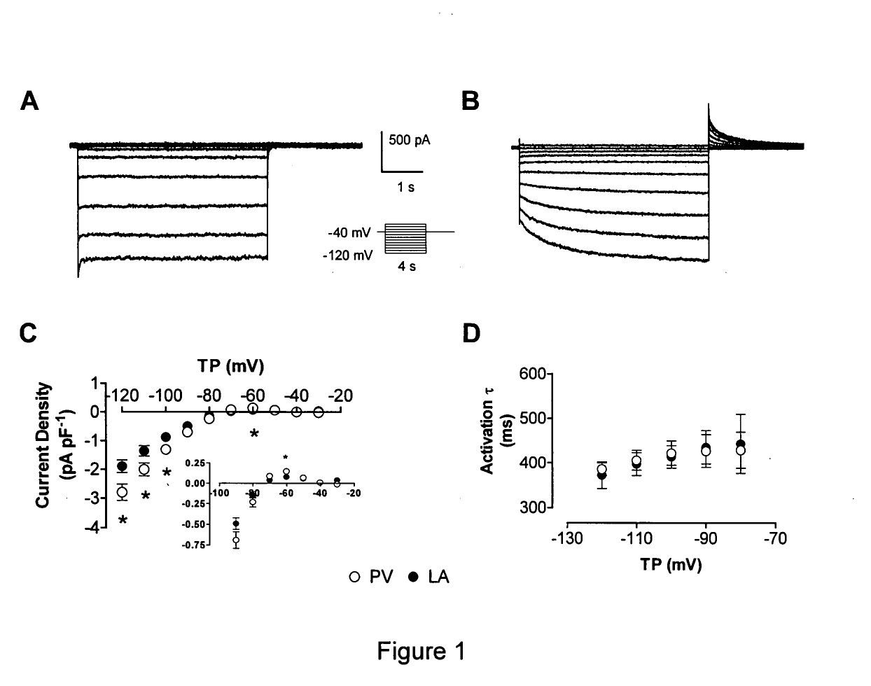

Upon voltage steps from −40 mV, 25% of LA and PV cardiomyocytes showed instantaneous inward currents with small inactivating components and strong inward rectification typical of I_{K1} (FIG. 1A). In the remaining 75% of cells, we observed time-dependent inward currents that activated over several seconds upon hyperpolarization, with outward tail currents upon repolarization (FIG. 1B). The instantaneous current component present immediately upon resolution of the capacitive current were quantified, as well as the slow time-dependent component, which was identified with I_{KH}. When the term I_{KH} is used without additional qualification in this document, it refers to the slowly time-dependent component. The slowly activating time-dependent current reversed at −72 mV (−84 mV with correction for the junction potential, compared to the calculated Nernst potential of −84.9 mV) and showed strong inward rectification (FIG. 1C). I_{KH} was greater in PV cardiomyocytes (e.g. average at −120 mV: −2.8±0.3 in PV versus-1.9±0.2 pA pF−1 in LA, n=27 and 26, respectively, P<0.01), for both inward and outward (inset of FIG. 1C) components. Current activation kinetics were well-fitted by mono-exponential functions, with time constants decreasing at more negative potentials and no significant kinetic differences between LA and PV cardiomyocytes (FIG. 1D).

Voltage Dependence of Activation

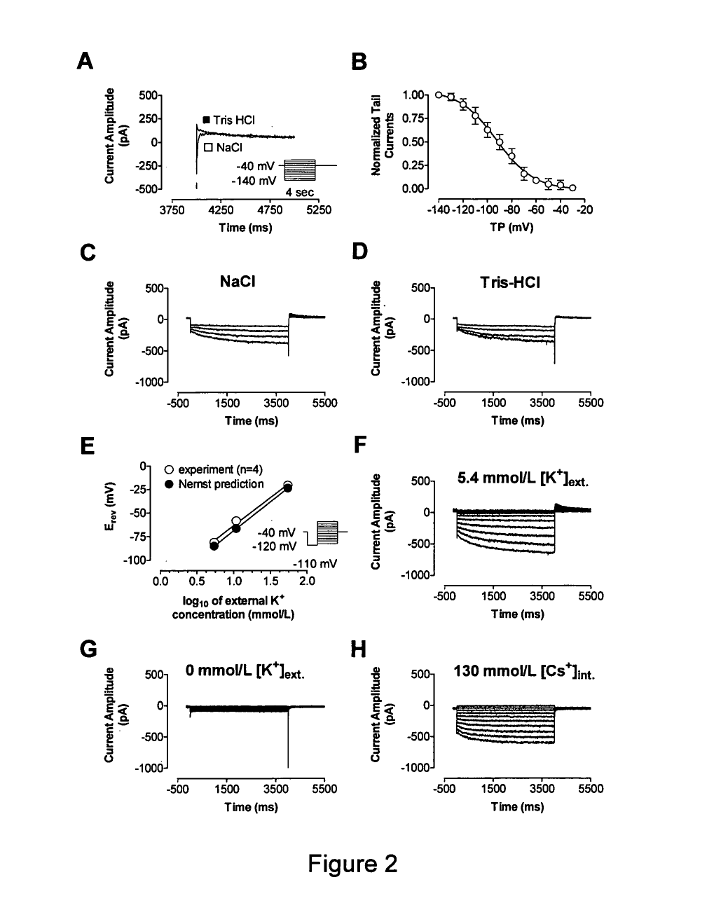

Tail currents recorded upon steps to −40 mV after hyperpolarization to negative potentials were contaminated by activating Na⁺ current (I_{Na}, FIG. 2A). Replacement of extracellular NaCl with equimolar Tris-HCl eliminated the inward I_{Na} component without altering the outward current tail (FIG. 2A). For example, I_{KH} tail amplitude (determined by back-extrapolation to the onset of the pulse to −40 mV) following a hyperpolarization to −120 mV was −185±53 pA before and −182±62 pA after Tris-HCl substitution (n=5 cells studied under both conditions, P=non significant). Tail-current density at −40 mV was a function of prepulse potential (FIG. 2B), suggesting voltage-dependent activation with a half-activation voltage (V50) of −93±4 mV and a slope factor of −15.2±2.2 (Boltzmann-distribution fit). Replacement of extracellular Na+ with Tris failed to significantly alter hyperpolarization-induced inward current, as illustrated in FIGS. 2C and D. In five cells studied in this fashion, I_{KH} at −120 mV averaged −3.1±0.9 and −3.0±0.9 pA pF⁻¹ (P=n.s.) in the presence and absence of extracellular Na⁺, respectively. As an additional approach to analysing activation voltage dependence, I_{KH} current-voltage data obtained as shown in FIG. 1C was evaluated according to the relationship:
A_{V}=I_{V}/Iₘₐₓ(V−Vᵣₑᵥ)

where a_{V} and I_{V} are the activation variable and activating I_{KH} amplitude at voltage V, Iₘₐₓ is I_{KH} amplitude at the most negative potential and Vᵣₑᵥ is the reversal potential (based on tail current analyses described below). The V50 provided by this approach averaged −87±3 mV (n=10 cells), not significantly different from the result obtained with tail current analysis. Tail currents were well-fitted by bi-exponential functions, with time constants at −40 mV averaging 243±15 and 1741±146 ms and the slow component averaging 39±2% of the total.

FIG. 2 illustrates the following data. Panel A shows tail currents recorded at repolarization to −40 mV after hyperpolarization to negative potentials (voltage protocol in inset) showed contamination by I_{Na} activated upon depolarization from negative test potentials back to the holding potential of −40 mV. Recordings upon returning to −40 mV after a hyperpolarization to −140 mV in one cell are shown before (□) and after (▪) substitution of extracellular sodium with Tris-HCl. Removal of Na+ eliminated the fast inward Na+ transient but did not affect the tail current. Panel B shows tail currents recorded in Na+o-free solution, normalized to values at test potential of −140 mV. Half-activation voltage obtained with Boltzmann fits to normalized tail currents was −93.1±4.6 mV, slope factor −15.2±2.2 (n=5 cells). Panels C and D show that removal of external sodium did not affect time-dependent inward IKH. A family of recordings obtained in the same cell with test pulses to −120, −100, −90 and −80 mV before (C) and after (D) substitution of NaCl by equimolar Tris-HCl is shown (similar results were obtained in 5 PV cardiomyocytes). Panel E shows the determination of tail current reversal (voltage protocol in inset) at various external K+ concentrations. The reversal potential became more positive as [K+]ₒ increased. Linear regression (◯) yielded a shift in mean Eᵣₑᵥ of 55.7±2.4 mV decade⁻¹ change in [K+]ₒ, with a correlation coefficient of 0.99 (n=4), and was in agreement with the prediction by the Nernst equation (37° C.) for a K+ conductance (61.5 mV decade-1, •). Examples of recordings before (F) and after (G) removal of external K+ in the same cell are shown, with K+ removal eliminating I_{KH} in this and 2 other cells tested. Removal of internal K+ and equimolar substitution with Cs⁺ (20 mM CsCl and 110 mM CsAsp) led to disappearance of the outward tail currents (n=3, H). [K+]ₒ, external potassium concentration; TP, test potential.

K⁺ Dependence and Inhibition by Ba²⁺ and Cs⁺

The reversal potential of the time-dependent component (FIG. 1C: −72.7±1.6 mV, n=6; −84 mV after correction for junction potential) was indicative of high K⁺ selectivity. With increasing [K⁺]ₑₓₜ., tail currents reversed at increasingly positive potentials (FIG. 2E). Tail current reversal potentials were quantified by linear regression of tail currents against voltage, based on data elicited with the protocol shown in the inset in FIG. 2E. A 10-fold change in external K⁺ led to a 55.7±2.4 mV decade⁻¹ shift in Eᵣₑᵥ (n=4, FIG. 2E) compared to 61.5 mV decade-1 predicted by the Nernst equation for a K+-specific current at 37° C. FIGS. 2F and G show I_{KH} recorded in one PV cell before and after exposure to nominally K+-free extracellular solution. In this and four other cells studied in the same fashion, elimination of extracellular K⁺ strongly suppressed I_{KH}. When I_{KH} was recorded with K⁺-free pipette solution (K⁺ replaced by Cs⁺), inward current remained but outward tail currents were absent (n=3, FIG. 2H).

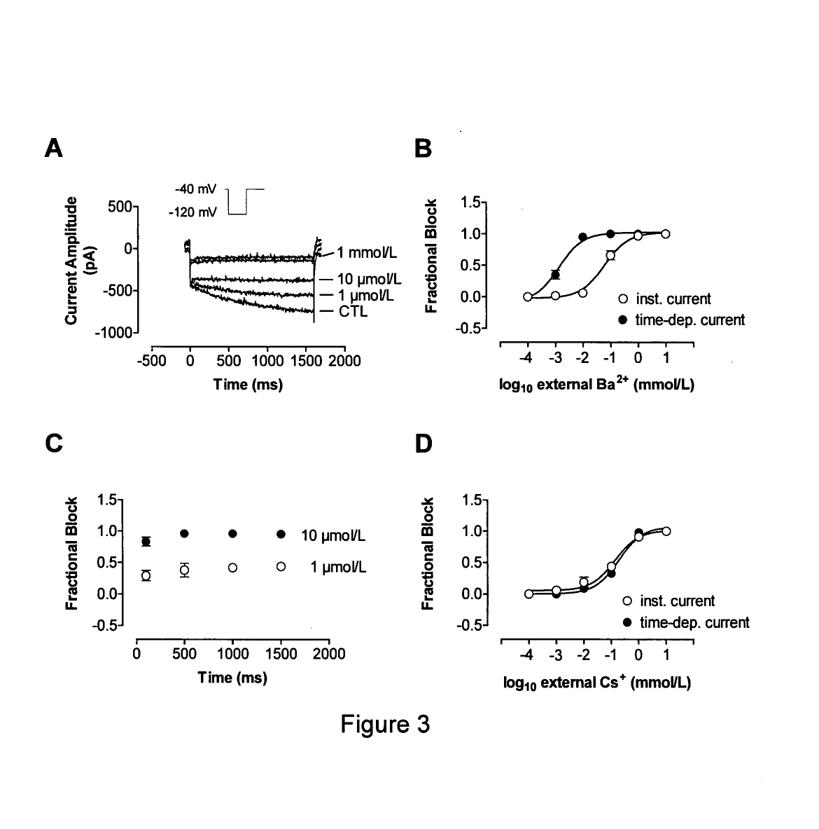

FIG. 3 illustrates the response of I_{KH} to extracellular Ba²⁺ and Cs⁺. Ba²⁺ inhibited I_{KH} appreciably at a concentration of 1 μM in most cells and full inhibition was generally seen at 10 μM (FIG. 3A). Mean concentration-response data for inhibition of the instantaneous component and time-dependent I^{KH} are shown in FIG. 3B. Ba²⁺ inhibited time-dependent I_{KH} more potently than the instantaneous current (IC50, 76.0±17.9 μM for instantaneous current versus 2.0±0.3 μM for I_{KH} at −120 mV, n=8 cells each, P<0.01). FIG. 3C shows block of I_{KH} as a function of time during pulses to −120 mV in four cells. The time-dependent current was calculated at each time point as the difference between the current immediately following hyperpolarization and the current level at the time point indicated. Fractional inhibition was calculated for each time point as the time-dependent current under control conditions minus the time-dependent current in the presence of Ba²⁺, divided by the time-dependent current under control conditions. Block showed minimal time dependence, suggesting that the difference in IC50 between instantaneous and time-dependent current is more likely to be due to intrinsic differences in sensitivity of different currents to block by Ba²⁺ than to a time-dependent blocking mechanism. FIG. 3D shows the response of instantaneous current and I^{KH} to Cs⁺. Both were highly and equally sensitive: at −120 mV, IC50 values averaged 139.0±34.2 versus 184.0±18.2 μM for instantaneous and time-dependent current, respectively (n=6 cells each, P=n.s.).

Response to Pharmacological Interventions

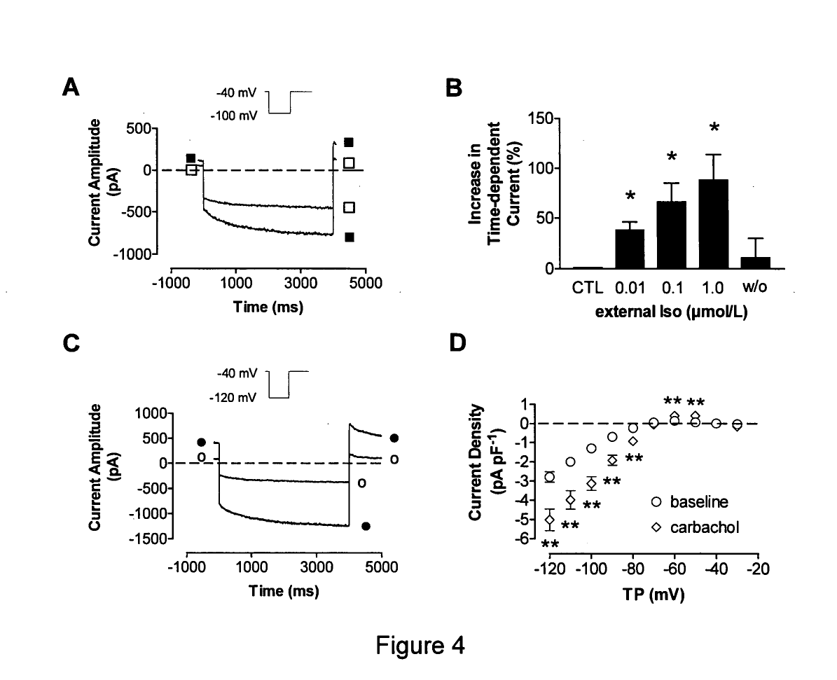

Isoproterenol was applied extracellularly at concentrations of 10, 100 and 1000 nM. FIG. 4A shows the effect of 1 μM isoproterenol on I_{KH} in a PV cell. A clear and reversible increase was seen, with steady-state effects achieved rapidly (within 2 min). Mean current density at −100 mV increased from −0.9±0.1 pA pF⁻¹ (control) to −1.2±0.1 pA pF⁻¹ in the presence of 10 nM isoproterenol, −1.4±0.2 pA pF⁻¹ with 100 nM isoproterenol and −1.5±0.1 pA pF⁻¹ with 1000 nM isoproterenol (n=14 cells, P<0.05 versus control for each concentration). Washout returned mean current amplitude to −0.9±0.2 pA pF⁻¹. Mean percentage changes from baseline at each isoproterenol concentration and upon washout are shown for PV cells in FIG. 4B. Isoproterenol also had concentration-dependent effects on holding current, which increased from 44±11 pA pF−1 under control conditions to 65±14 pA pF−1 (59±14% increase) at 10 nM isoproterenol, 70±13 pA pF−1 (89±33% increase) at 100 nM isoproterenol and 87±14 pA pF−1 (144±37% increase) at 1000 nM (P<0.05 versus control for all). Holding current changes were also reversible after washout.

The inward rectification of I_{KH} led to considering the possibility that it might be related to cholinergic K⁺ current, and its response to the muscarinic agonist carbachol was therefore tested. Carbachol (1 μM) strongly increased both instantaneous and time-dependent current, as illustrated by the response of a PV cell illustrated in FIG. 4C. Mean time-dependent I_{KH} density-voltage relations recorded before and after carbachol in nine PV cells are shown in FIG. 4D. Carbachol increased IKH by an average of 188±54% at voltages between −120 and −90 mV. I_{KH} in both PV and LA cells responded to carbachol (e.g. currents at −120 mV increased to −5.0±0.6 and −4.9±1.1 pA pF⁻¹ from −2.6±0.5 and −1.9±0.4 pA pF⁻¹ in PV and LA, respectively, P<0.01 versus baseline, n=9 cells each). Blockade of muscarinic receptors with 200 nM atropine completely abolished the effect of carbachol (data not shown). Atropine itself had no effect on I_{KH} in the absence of carbachol.

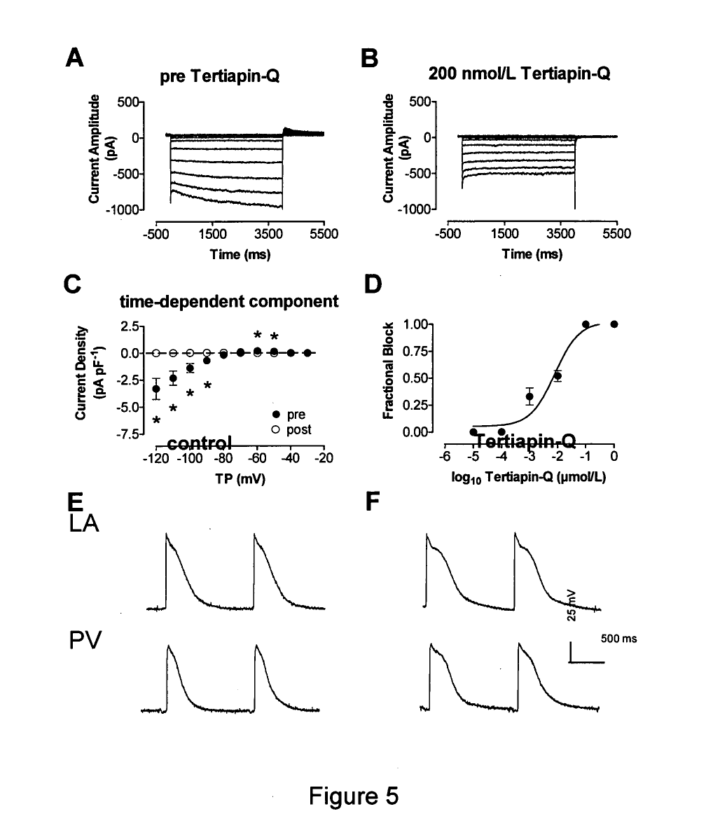

In view of the response to carbachol, the possibility that channels composed of Kir3 subunits carry I_{KH} was considered. Tertiapin-Q is a 22-amino acid peptide synthesized from honeybee venom that blocks Kir3-based currents at nanomolar concentrations without affecting Kir2 currents (Jin & Lu, 1999), and has been found to block acetylcholine-dependent current in the heart in a highly selective fashion (Drici et al. 2000). FIG. 5 shows a family of currents recorded in a PV cardiomyocyte under control conditions (A) and after exposure to 200 nM tertiapin-Q (B). The inhibitory effect was relatively completely reversible upon washout of the drug (data not shown). Tertiapin-Q relatively completely and relatively reversibly abolished time-dependent I_{KH}. FIG. 5C shows mean current-voltage relations for the time-dependent component of the current in seven PV cardiomyocytes before and after tertiapin-Q. Whereas the time-dependent current was eliminated after 1 μM tertiapin-Q application, mean instantaneous current decreased by 25%, a change that was not statistically significant. These data indicate that the time-dependent component is entirely tertiapin-Q sensitive, whereas a statistically non-significant minority of the instantaneous current is blocked by tertiapin-Q, supporting the notion that the instantaneous component is largely carried by a conductance (probably I_{K1}) that is distinct from I_{KH}. Mean concentration-response data for the effect of tertiapin-Q on time-dependent IKH at −120 mV in four PV cardiomyocytes are shown in FIG. 5D, and provide an IC50 of 10±2 nM, close to the reported IC50 for Kir 3.1/3.4 currents of 8 nM (Jin & Lu, 1999).

Having shown this effect of tertiapin-Q on I_{KH}, the application of 1 μM isoproterenol was repeated in the presence of 200 nM tertiapin-Q in order to evaluate the potential contribution of currents other than I_{KH} (such as I_{f}) to the enhancement of time-dependent hyperpolarization-activated current. In the presence of tertiapin-Q, isoproterenol failed to elicit significant hyperpolarization-activated current. For example, mean end-pulse current at −120 mV averaged −2.84±0.89 pA pF⁻¹ before tertiapin-Q in 4 cells, versus 0.09±0.01 pA pF⁻¹ (P<0.05) after tertiapin-Q and 0.20±0.07 pA pF⁻¹ in the same cells in the presence of tertiapin-Q and isoproterenol (P=not significant versus pre-isoproterenol).

Role of I_{KH} in AP Repolarization

Given the high selectivity of tertiapin-Q for Kir3 channels and its potent inhibition of I_{KH}, the effect of tertiapin-Q on repolarization of LA and PV APs was evaluated in multicellular canine atrial preparations in the presence of 200 nM atropine to prevent any contribution of endogenously released acetylcholine. FIGS. 5E and F show representative APs before and after 100 nM tertiapin-Q in LA and PV. Mean AP duration characteristics are provided in Table 2, and indicate that tertiapin-Q significantly prolonged AP duration in both regions.

Potential Role of G Proteins in I_{KH} Regulation

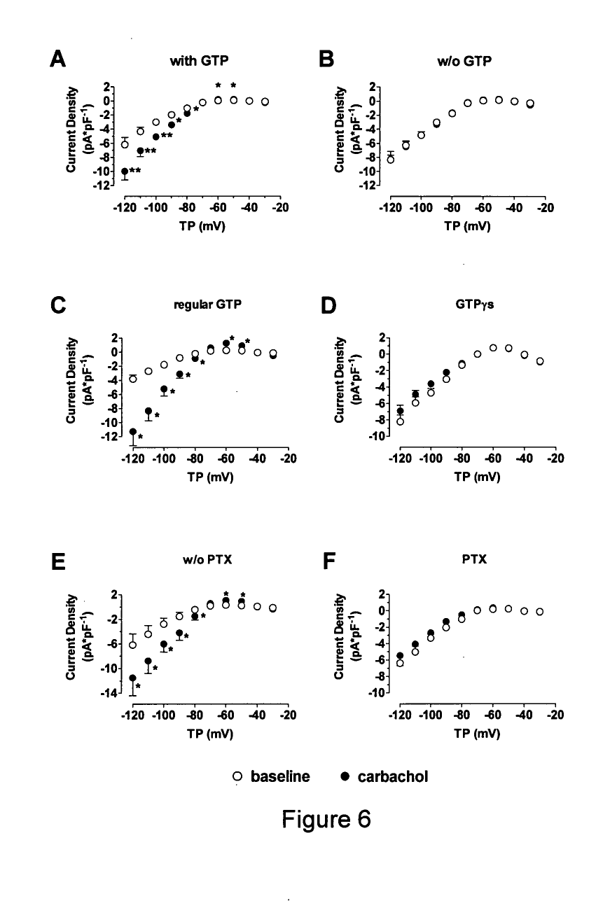

In the light of a possible contribution of Kir3 channels to I_{KH} current, the effects of a variety of interventions targeting G proteins were studied. In each case, experiments were performed in one cell under control conditions followed by another cell from the same batch studied in the presence of an intervention, in order to exclude data contamination by inter-day and inter-isolate differences in IKH amplitude and response. When GTP was not included in the pipette solution, basal IKH was not altered. However, in the absence of pipette GTP the addition of carbachol failed to further activate IKH (FIGS. 6A and B). For example, at −120 mV current density in cells patched with GTP-free pipettes averaged −8.3±0.7 versus −8.3±1.1 pA pF⁻¹ (P=n.s., n=4) before and after 1 μM carbachol, respectively. In the same batches of cells studied on the same days with GTP-containing pipettes, I_{KH} increased from −6.2±1.0 to −10.0±1.2 pA pF⁻¹ (P<0.05, n=4) upon exposure to carbachol.

Then, the effect of substituting the non-hydrolysable analogue GTPS for GTP in the pipette was investigated. As illustrated in FIGS. 6C and 6D, the inclusion of GTPS significantly increased I_{KH} in the absence of cholinergic stimulation compared to current recorded with pipettes containing regular GTP (e.g. at −120 mV from −3.8±0.6 to −8.2±0.8 pA pF⁻¹, P<0.05, n=6 cells each). In addition, GTPS fully prevented any further I_{KH} response to carbachol, suggesting that I_{KH} was already maximally activated in the presence of GTPS.

Preincubation with 1.5 mg I⁻¹ PTX also attenuated the effect of carbachol (FIGS. 6E and F). For example, at −120 mV carbachol increased IKH density from −6.2±1.8 to −11.5±1.8 pA pF⁻¹ in cells incubated without PTX at 37° C. for >9 h (P<0.05 for carbachol effect, n=6). In cells preincubated with PTX at the same temperature and time, IKH density averaged −6.4±0.8 pA pF⁻¹ before carbachol and −5.5±1.2 pA pF−1 in the presence of carbachol at −120 mV (P=not significant, n=6). The response of I_{KH} tail current to carbachol was significantly reduced in PTX-treated cells, but not abolished. For example, following hyperpolarization to −120 mV tail currents averaged 2.1±0.7 and 8.4±1.6 pA pF⁻¹ (P<0.01) before and after carbachol under PTX-free conditions and 1.8±0.6 and 3.4±0.9 pA pF⁻¹ (P<0.01) before and after carbachol in PTX-preincubated cells (n=6 for each). Overall, carbachol increased I_{KH} tail currents following a pulse to −120 mV by 538±170% in cells incubated without PTX versus 127±37% (P<0.05 versus change in absence of PTX) in PTX-treated cells.

Potential Role of I_{KH} in Atrial Tachycardia-Induced Remodelling

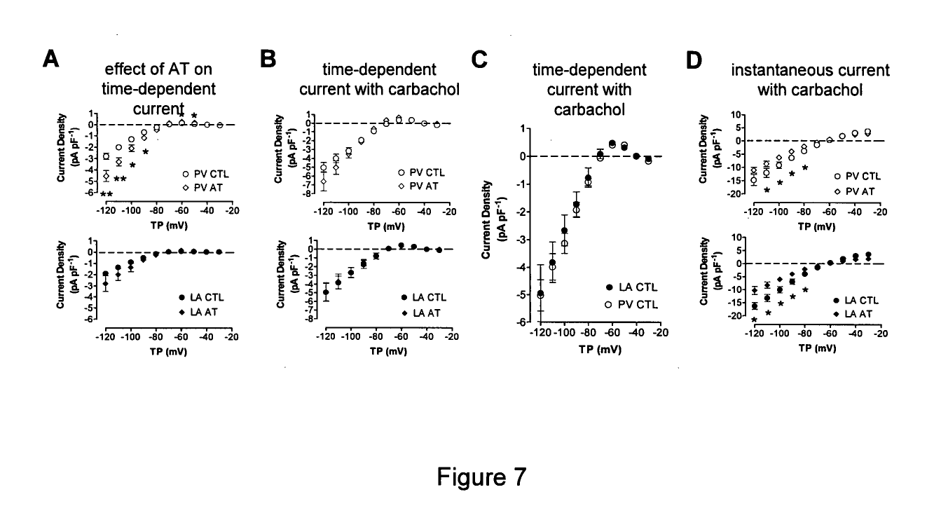

FIG. 7A shows mean±Standar Error of Mean (S.E.M.) I_{KH} density-voltage relations in control dogs and dogs subjected to 7 day atrial tachycardia (AT) for LA and PV cardiomyocytes. I_{KH} was increased modestly in LA cardiomyocytes. Larger increases in I_{KH} were seen in PV cells.

I_{KH} was not significantly affected by AT remodelling when measured in the presence of 1-μM carbachol (FIG. 7B), in contrast to the up-regulation observed in the absence of carbachol (FIG. 7A). These results suggest that maximally available I_{KH} is similar in the absence versus presence of AT remodelling, and that regulatory factors may explain the differences observed in the absence of cholinergic stimulation. Similarly, in the presence of 1 μM carbachol, time-dependent IKH densities were not significantly different between control LA and PV cells (FIG. 7C), in contrast to the higher PV IKH density in the absence of carbachol, again consistent with a prime role for regulatory factors in the PV−LA IKH difference. In contrast, the instantaneous component recorded in the presence of 1 μM carbachol was significantly reduced by AT remodelling in both LA and PV cells (FIG. 7D).

Possible Molecular Basis for I_{KH}

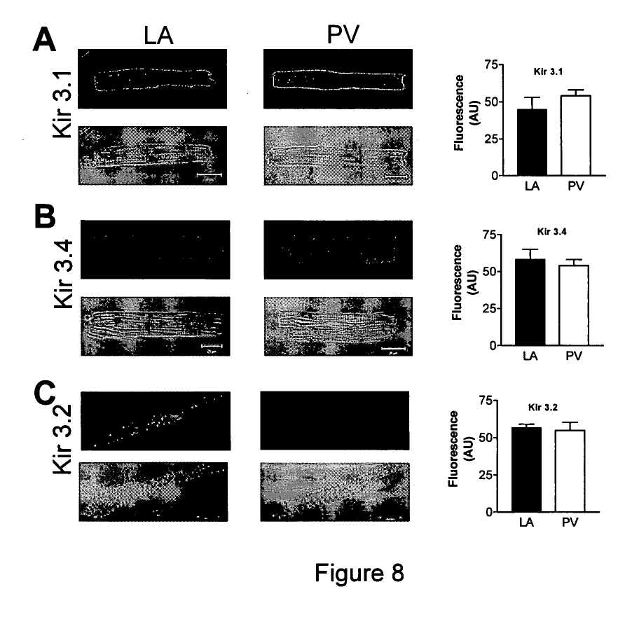

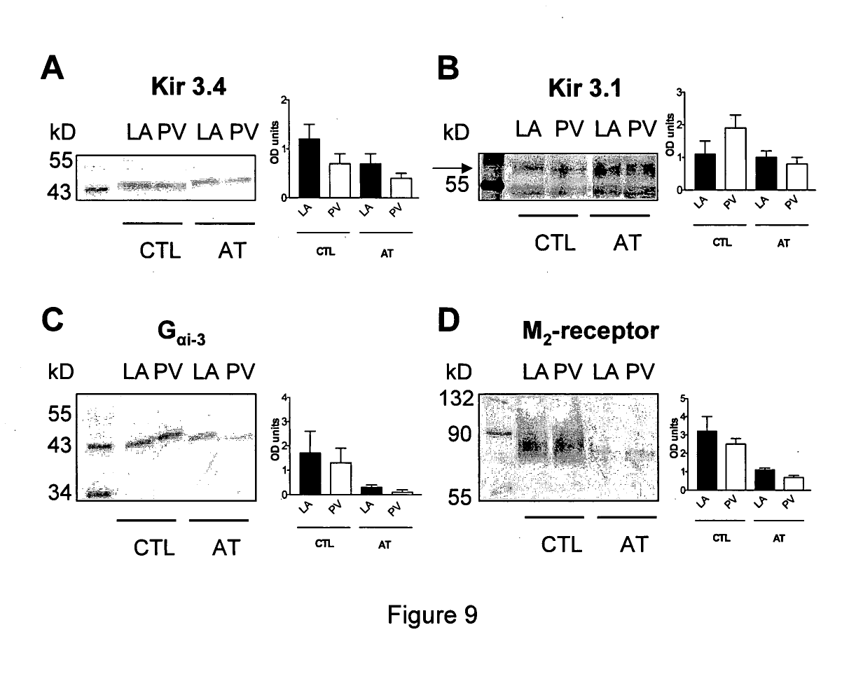

The expression of Kir3.1, 3.2 and 3.4 channel subunits was evaluated in isolated LA and PV cardiomyocytes with Western blot and semiquantitative immunohistochemical methods. Immunohistochemical studies confirmed the presence of Kir3.1 and 3.4 on isolated LA and PV cardiomyocytes, with clear membrane staining (FIGS. 8A and B). Kir3.2 staining was fainter and no outer membrane distribution was observed (FIG. 8C). Quantitative analysis of immunofluorescence indicated no significant differences between LA and PV cardiomyocytes in Kir3 subunit immunofluorescence intensity (right panels). Kir3 subunit expression as measured by Western blotting of isolated cardiomyocyte membrane preparations was not significantly different in LA versus PV (FIGS. 9A and B). Atrial tachypacing reduced Kir3.4 expression, but did not significantly affect Kir3.1. M2 muscarinic receptor and inhibitory G protein (Gi) expression was down-regulated by atrial tachycardia-induced remodelling (FIGS. 9C and D), consistent with the decreased response of the instantaneous component to carbachol shown in FIG. 7C.

Discussion

Major Findings

A time-dependent inwardly rectifying K⁺-current, I_{KH}, was characterized in canine atrial cardiomyocytes. I_{KH} sensitivity to Ba²⁺ is instantaneous rather than time dependent, favouring conductance by Kir3 channels over Kir2 (Yamada et al. 1998). I_{KH} sensitivity to tertiapin-Q resembles that seen with Kir3.1/3.4 channels (Jin & Lu, 1999), pointing to possible constitutive, agonist-independent Kir3.1/3.4 activity. I_{KH} is subject to modulation by important endogenous neurotransmission systems (adrenergic and cholinergic), and by a recognized atrial arrhythmogenic intervention (atrial tachycardia-induced remodelling).

Possible Molecular Basis

I_{KH} is an inwardly rectifying, highly K⁺-selective conductance sensitive to Ba²⁺, properties compatible with several inward-rectifying Kir subunits. Tertiapin-Q is a highly selective inward-rectifier K⁺ channel blocker that inhibits Kir1 channels with an IC50 of 2 nM and Kir3.1/3.4 with an IC50 of 8 nM, but has minimal effects on Kir2.1 channels at micromolar concentrations (Jin & Lu, 1999). Acetylcholine-dependent K+ current (I_{KACh}) in native cells, based on Kir3.1 and 3.4 subunit heteromers, is suppressed by tertiapin-Q with IC50 values ranging from 8 to 30 nM (Drici et al. 2000; Kitamura et al. 2000), concentrations with no effect on I_{Kr}, I_{Ks}, Iₜₒ, I_{K1} or I_{KATP} (Drici et al. 2000; Kitamura et al. 2000). The action of tertiapin-Q on I_{KACh} is independent of muscarinic receptor activation state (Yamada, 2002). I_{KH}-like kinetics have been noted for I_{KACh} in human atrial myocytes (Heidbuchel et al. 1987) and Kir3.1/3.4 channels activated by Gβ subunits (Reuveny et al. 1994). Kir1.1 subunits carry tertiapin-sensitive currents (Jin & Lu, 1999) and are detectable in PV cardiomyocytes (Michelakis et al. 2001); however, Kir1-based currents lack I_{KH} kinetics (Schuck et al. 1994) and are more sensitive to tertiapin-Q than Kir3 current (Jin & Lu, 1999), I_{KACh} (Drici et al. 2000; Kitamura et al. 2000) or I_{KH}. All of these observations suggest that I_{KH} is carried by constitutive Kir3 subunit activity.

PV expression of Kir3.1 and Kir3.4 proteins was not different from LA (FIGS. 8 and 9), suggesting that larger PV I_{KH} may be due to regulatory factors, rather than differences in ion channel expression. Signaling events besides direct Gβ modulation of Kir3 channels may be important for determining basal and agonist-stimulated current (Yamada et al. 1998). Kir3-channel activity is increased by protein kinase A (PKA) and inhibited by phosphatases (Mullner et al. 2000). Phosphatidylinositol phosphates increase current through Kir3 channels and are regulated by Gq-mediated signalling (Kobrinsky et al. 2000). Channel activity is regulated by intracellular sodium and chloride (Mirshahi et al. 2003) and by extracellular and intracellular pH (Mao et al. 2003). Stretch also inhibits Kir3 channels (Zhang et al. 2003). Ca2+-calmodulin facilitates GTPase activity of regulators of G protein signalling (RGS) proteins by suppressing inhibitory effects of phosphatidylinositol-3,4,5-trisphosphate (PIP3) on RGS4 activity (Ishii et al. 2002). G protein-regulated K+ currents showed time dependence like I_{KH}, suggesting that similar complex lipid-protein interactions may regulate I_{KH} kinetics and function. Such regulation may be important in maintaining basal I_{KH} activity, as neither PTX nor absence of GTP affected I_{KH} in the absence of carbachol. Alterations in these signalling pathways may occur during atrial tachypacing and may lead to increased basal I_{KH}. Further experiments are needed to define the exact mechanisms of I_{KH} regulation.

Potential Significance

Tertiapin-Q prolonged AP duration recorded with standard microelectrode techniques from multicellular LA and PV preparations in the presence of atropine to exclude contributions from endogenous acetylcholine, pointing to a potentially significant role for I_{KH} in LA and PV cardiomyocyte repolarization. Since atrial tachycardia-induced remodelling increases I_{KH}, it may contribute to AF-promoting action potential abbreviation caused by persistent atrial tachyarrhythmias (Yue et al. 1997; van der Velden et al. 2000).

The autonomic nervous system (parasympathetic and sympathetic) is known to contribute to atrial arrhythmogenesis. β-Adrenergic stimulation hyperpolarizes atrial myocytes (Boyden et al. 1983); however, isoproterenol typically inhibits I_{K1} (Koumi et al. 1995; Zhang et al. 2002). The increase in I_{KH} caused by 1′-adrenergic stimulation shown here is a potential contributor to AF promotion and atrial myocyte hyperpolarization resulting from adrenergic stimulation. I_{KH} is also a candidate to participate in cholinergic AF promotion. Atrial tachycardia-induced remodelling is a significant factor in clinical AF (Nattel, 2002). Ionic current changes that may contribute to remodelling-induced APD-abbreviation include decreased I_{Ca} and increased I_{K1} (Yue et al. 1997; Bosch et al. 1999; Dobrev et al. 2001). The present findings add I_{KH} up-regulation as a potential contributor to atrial-tachycardia induced AP abbreviation. The larger I_{KH} in PV versus LA cells after tachycardia-induced remodelling (FIG. 7) suggests that I_{KH} may contribute to the role of PVs in AF maintenance (Wu et al. 2001).

Our study suggests that β-adrenoceptor activation can stimulate Kir3-based channels in cardiomyocytes. Kir3 channels are opened by the Gβ heterodimer in response to Gi activation by M2 muscarinic receptors (Lim et al. 1995). It was believed that β-adrenoceptors were incapable of modulating these currents in native tissues (Trautwein et al. 1982). Some investigators found an increase in IKACh with isoproterenol application (Kim, 1990; Sorota et al. 1999; Mullner et al. 2000). Others attributed the effect of isoproterenol on IKACh to isoproterenol-activated IK,ATP (Wang & Lipsius, 1995). The present study suggests that β-adrenoceptor stimulation of Kir3-based current occurs in LA and PV cells, and that the organization of signalling may therefore be cell-type dependent.

### EXAMPLE 2

Adult dogs were divided into control and AT groups. AT dogs were initially anesthetized with diazepam (0.25 mg/kg IV), ketamine (5.0 mg/kg IV), and halothane (1% to 2%) for transvenous insertion of right ventricular (RV) and right atrial (RA) unipolar pacing leads connected to ventricular and atrial pacemakers implanted in the neck. Pacing began 24 hours after pacemaker implantation. AV block was created by radiofrequency catheter ablation and the RV demand-pacemaker was programmed to 80 bpm. The RA was then tachypaced (400 bpm) for 7-13 days.

On study days, dogs were anesthetized with pentobarbital (30 mg·kg⁻¹ I.V.) and artificially ventilated. Hearts and adjacent lung tissue were excised via a left thoracotomy and immersed in oxygenated Tyrode solution. For cell isolation, the proximal circumflex coronary artery was cannulated and distal ends of PV myocardial sleeves were marked with silk thread prior to enzyme perfusion with collagenase (100 U mL⁻¹, Worthington, type II), to facilitate identification after enzymatic digestion. PVs were well-perfused and single cardiomyocytes were isolated from all PVs. After isolation, cells were stored at 4° C. and studied within 12 hours.

For standard-microelectrode experiments and atrial-tachyarrhythmia induction, intact tissue preparations including the LA and adjacent PVs were mounted in a Plexiglas chamber and perfused via the left circumflex coronary artery with oxygenated Kreb's solution at 36±0.5° C. Fine-tipped microelectrodes (resistance 15-20 MΩ when filled with 3-mol/L KCL) coupled to a high-input impedance amplifier were used to record Action Potentials.

For atrial tachyarrhythmia induction, single S2-extrastimuli following 15 S1-stimuli at basic cycle lengths (CLs) of 150-400 ms with a 5-second pause to observe the response were used. All stimuli were twice-threshold, 2-ms pulses.

For single cardiomyocytes, currents were recorded with whole-cell patch-clamp at 36±0.5° C. Borosilicate-glass electrodes had tip resistances between approximatively 1.5 and 3.0 MΩ when filled. To control for cell-size variability, currents are expressed as densities (pA/pF).

As mentioned hereinabove AT pacing increased I_{KH} at −110 mV from −2.2±0.6 pA/pF (control) to −3.8±0.7 pA/pF (AT) in LA cardiomyocytes, and the 100 nM TQ-sensitive component increased from −1.7±0.5 (control) to −2.8±0.5 (AT) pA/pF. The TQ-sensitive component is obtained by subtracting the current in the presence of TQ from the current without TQ.

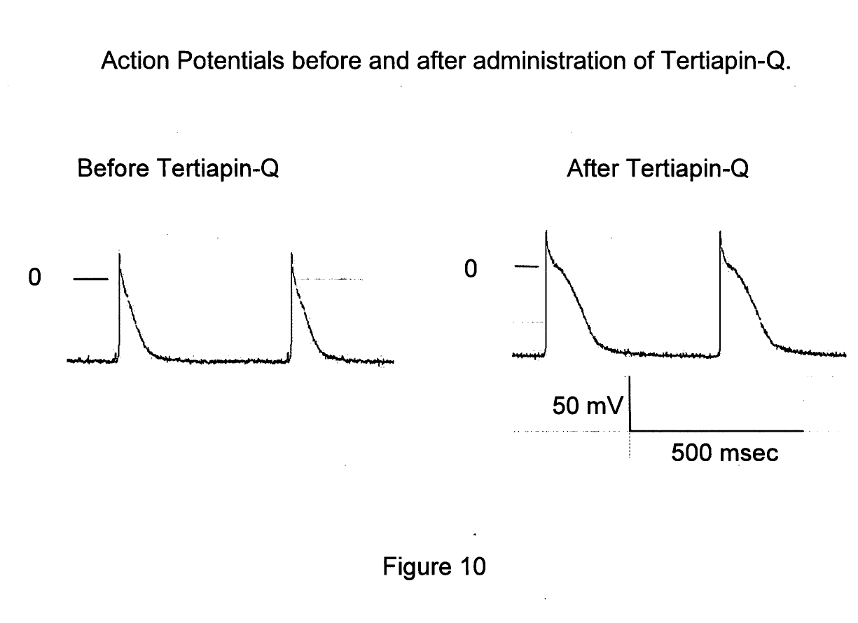

FIG. 10A, shows an example atrial tachycardia action potentials prior to treatment with tertiapin-Q, while FIG. 10B shows an example an atrial tachycardia action potentials further to treatment with tertiapin-Q. Comparing FIG. 10A to FIG. 10B clearly shows that tertiapin-Q greatly increases action potential duration.

Table 1 quantifies this increase in duration by presenting the time required for action potentials in 8 dogs to reach 90%, 50% and 25% repolarization. Results are averages over 24 and 23 action potentials respectively for the pre-TQ data and the post-TQ data.

Prolonged atrial tachyarrhythmias could be induced with single extrastimuli at varying cycle lengths in AT-remodeled, but not control, preparations. This confirms the well-known fact that AT-remodeling is a good model, at least in some cases, for cardiac changes leading to AF.

In AT-remodeled preparations, mean tachyarrhythmia duration was 11602±604 ms (n=81, 8 dogs), with a cycle length of 108±0.3 ms. Tachyarrhythmia duration was decreased significantly by 100 nM TQ, to 520±6 ms (n=66, 8 dogs, P<0.05), and tachyarrhythmia cycle length increased to 179±0.8 ms (P<0.001).

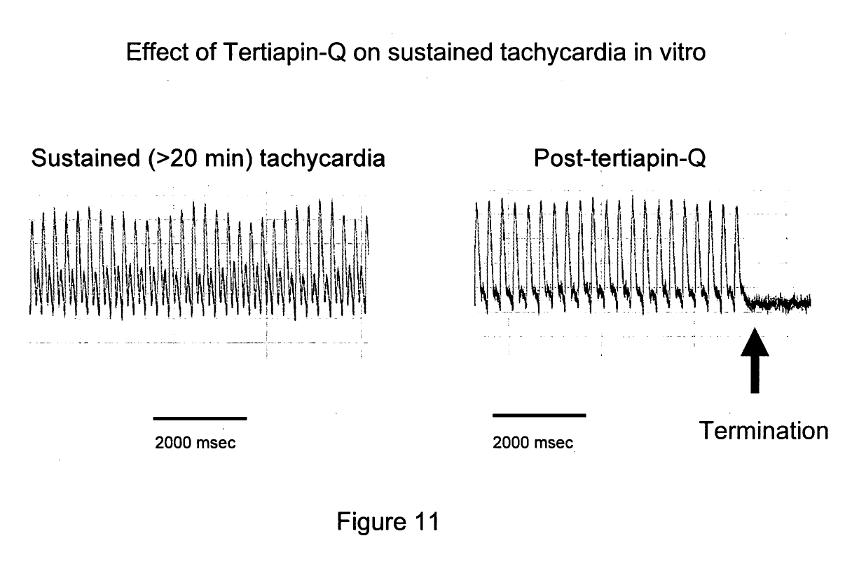

In 2 cases, tachyarrhythmia lasted uninterrupted for more than 20 minutes; TQ administration to tissue preparations terminated arrhythmia within 4 minutes in both. FIG. 11 illustrates such a termination of tachyarrhythmia further to the administration of TQ.

Although the present invention has been described hereinabove by way of preferred embodiments thereof, it can be modified, without departing from the spirit and nature of the subject invention as defined in the appended claim.

TABLE 1
time required for action potentials in 8 dogs to reach 90%, 50% and
25% repolarization. Results are averages over 24 and 23 action potentials
respectively for the pre-TQ data and the post-TQ data.

| 2 Hz | APD90 (ms) | APD50 (ms) | APD25 (ms) |
|---|---|---|---|
| Pre-TQ (n = 24, | 100.1 ± 2.6 | 44.4 ± 2.3 | 15.9 ± 1.7 |
| 8 dogs) | | | |
| TQ (n = 23, | 162.2 ± 4.2** | 87.4 ± 2.7** | 39.0 ± 2.5** |
| 8 dogs) | | | |

**P < 0.001 TQ vs. pre-TQ. APD90, APD50, APD25 = action potential duration to 90, 50, 25% repolarization respectively.

TABLE 2
time required for action potentials in Pulmonary vein (PV) and Left atrial
(LA) cardiomyocytes to reach 90%, 50% and 25% repolarization. Results are presented
under control conditions and after exposure to 200 nM tertiapin-Q.

| | Control | | | Tertiapin-Q | | |
|---|---|---|---|---|---|---|
| | APD90 | APD50 | APD25 | APD90 | APD50 | APD25 |
| | (ms) | (ms) | (ms) | (ms) | (ms) | (ms) |
| LA | 202.6 ± 2.6 | 130.2 ± 4.0 | 78.1 ± 2.8 | 242.7 ± 6.2* | 146.8 ± 3.2* | 84.7 ± 4.0 |
| PV | 190.4 ± 4.3† | 104.0 ± 3.8† | 71.3 ± 3.4 | 234.2 ± 9.9* | 126.8 ± 6.9* | 86.1 ± 5.0* |

### REFERENCES

Bosch R F, Zeng X, Grammer J B, Popovic K, Mewis C & Kuhikamp V (1999). Ionic mechanisms of electrical remodeling in human atrial fibrillation. Cardiovasc Res 44, 121-131.
Boyden P A, Cranefield P F & Gadsby D C (1983). Noradrenaline hyperpolarizes cells of the canine coronary sinus by increasing their permeability to potassium ions. J Physiol 339, 185-206.
Chen Y J, Chen S A, Chen Y C, Yeh H I, Chan P, Chang M S & Lin C I (2001). Effects of rapid atrial pacing on the arrhythmogenic activity of single cardiomyocytes from pulmonary veins: implication in initiation of atrial fibrillation. Circulation 104, 2849-2854.
DiFrancesco D (1993). Pacemaker mechanisms in cardiac tissue. Annu Rev Physiol 55, 455-472.
Dobrev D, Graf E, Wettwer E, Himmel H M, Hala O, Doerfel C, Christ T, Schuler S & Ravens U (2001). Molecular basis of downregulation of G-protein-coupled inward rectifying K(+) current (I(K,ACh) in chronic human atrial fibrillation: decrease in GIRK4 mRNA correlates with reduced I(K,ACh) and muscarinic receptor-mediated shortening of action potentials. Circulation 104, 2551-2557.
Drici M D, Diochot S, Terrenoire C, Romey G & Lazdunski M (2000). The bee venom peptide tertiapin underlines the role of I(KACh) in acetylcholine-induced atrioventricular blocks. Br J Pharmacol 131, 569-577.
Ehrlich J R, Cha T J, Zhang L, Chartier D, Melnyk P, Hohnloser S H & Nattel S (2003). Cellular electrophysiology of canine pulmonary vein cardiomyocytes: action potential and ionic current properties. J Physiol 551, 801-813.
Haissaguerre M, Jais P, Shah D C, Takahashi A, Hocini M, Quiniou G, Garrigue S, Le Mouroux A, Le Metayer P & Clementy J (1998). Spontaneous initiation of atrial fibrillation by ectopic beats originating from the pulmonary veins. N Engl J Med 339, 659-666.
Heidbuchel H, Callewaert G, Vereecke J & Carmeliet E (1992a). Activation of guinea pig atrial muscarinic K+ channels by nucleoside triphosphates in the absence of acetylcholine. J Cardiovasc Electrophysiol 3, 457-473.
Heidbuchel H, Callewaert G, Vereecke J & Carmeliet E (1992b). Membrane-bound nucleoside diphosphate kinase activity in atrial cells of frog, guinea pig, and human. Circ Res 71, 808-820.
Heidbuchel H, Vereecke J & Carmeliet E (1987). The electrophysiological effects of acetylcholine in single human atrial cells. J Mol Cell Cardiol 19, 1207-1219.
Ishii M, Inanobe A & Kurachi Y (2002). PIP3 inhibition of RGS protein and its reversal by Ca2+/calmodulin mediate voltage-dependent control of the G protein cycle in a cardiac K+ channel. Proc Natl Acad Sci USA 99, 4325-4330.
Jin W & Lu Z (1999). Synthesis of a stable form of tertiapin: a high-affinity inhibitor for inward-rectifier K⁺ channels. Biochemistry 38, 14286-14293.
Kim D (1990). Beta-adrenergic regulation of the muscarinic-gated K+ channel via cyclic AMP-dependent protein kinase in atrial cells. Circ Res 67, 1292-1298.
Kitamura H, Yokoyama M, Akita H, Matsushita K, Kurachi Y & Yamada M (2000). Tertiapin potently and selectively blocks muscarinic K(+) channels in rabbit cardiac myocytes. J Pharmacol Exp Ther 293, 196-205.
Kneller J, Ramirez R J, Chartier D, Courtemanche M & Nattel S (2002). Time-dependent transients in an ionically based mathematical model of the canine atrial action potential. Am J Physiol Heart Circ Physiol 282, H1437-H1451.
Kobrinsky E, Mirshahi T, Zhang H, Jin T & Logothetis D E (2000). Receptor-mediated hydrolysis of plasma membrane messenger PIP2 leads to K+-current desensitization. Nature Cell Biol 2, 507-514.
Koumi S, Wasserstrom J A & Ten Eick R E (1995). Beta-adrenergic and cholinergic modulation of inward rectifier K+ channel function and phosphorylation in guinea-pig ventricle. J Physiol 486, 661-678.
Li D, Fareh S, Leung T K & Nattel S (1999). Promotion of atrial fibrillation by heart failure in dogs: atrial remodeling of a different sort. Circulation 100, 87-95.
Lim N F, Dascal N, Labarca C, Davidson N & Lester H A (1995). A G protein-gated K channel is activated via beta 2-adrenergic receptors and G beta gamma subunits in Xenopus oocytes. J General Physiol 105, 421-439.
Mao J, Wu J, Chen F, Wang X & Jiang C (2003). Inhibition of G-protein-coupled inward rectifying K+ channels by intracellular acidosis. J Biol Chem 278, 7091-7098.
Michelakis E D, Weir E K, Wu X, Nsair A, Waite R, Hashimoto K, Puttagunta L, Knaus H G & Archer S L (2001). Potassium channels regulate tone in rat pulmonary veins. Am J Physiol Lung Cell Mol Physiol 280, L1138-L1147.
Mirshahi T, Jin T & Logothetis D E (2003). G beta gamma and KACh: old story, new insights. Sci STKE 194, PE32.
Mullner C, Vorobiov D, Bera A K, Uezono Y, Yakubovich D, Frohnwieser-Steinecker B, Dascal N & Schreibmayer W (2000). Heterologous facilitation of G protein-activated K(+) channels by beta-adrenergic stimulation via cAMP-dependent protein kinase. J General Physiol 115, 547-558.
Nattel S (2002). New ideas about atrial fibrillation 50 years on. Nature 415, 219-226.
Pappone C, Rosanio S, Oreto G, Tocchi M, Gugliotta F, Vicedomini G, Salvati A, Dicandia C, Mazzone P, Santinelli V, Gulletta S & Chierchia S (2000). Circumferential radiofrequency ablation of pulmonary vein ostia. A new anatomic approach for curing atrial fibrillation. Circulation 102, 2619-2628.
Reuveny E, Slesinger P A, Inglese J, Morales J M, Iniguez-Lluhi J A, Lefkowitz R J, Bourne H R, Jan Y N & Jan L Y (1994). Activation of the cloned muscarinic potassium channel by G protein beta gamma subunits. Nature 370, 143-146.
Schuck M E, Bock J H, Benjamin C W, Tsai T D, Lee K S, Slightom J L & Bienkowski M J (1994). Cloning and characterization of multiple forms of the human kidney ROM-K potassium channel. J Biol Chem 269, 24261-24270.
Sorota S, Rybina I, Du Yamamoto A & X Y (1999). Isoprenaline can activate the acetylcholine-induced K+ current in canine atrial myocytes via Gs-derived betagamma subunits. J Physiol 514, 413-423.
Trautwein W, Taniguchi J & Noma A (1982). The effect of intracellular cyclic nucleotides and calcium on the action potential and acetylcholine response of isolated cardiac cells. Pflugers Arch 392, 307-314.

van der Velden HMWZL, Wijffels M C, van Leuven C, Dorland R, Vos M A, Jongsma H J & Allessie M A (2000). Atrial fibrillation in the goat induces changes in monophasic action potential and mRNA expression of ion channels involved in repolarization. J Cardiovasc Electrophysiol 11, 1262-1269.
Wang Y G & Lipsius S L (1995). Beta-adrenergic stimulation induces acetylcholine to activate ATP-sensitive K+ current in cat atrial myocytes. Circ Res 77, 565-574.
Wu T J, Ong J J, Chang C M, Doshi R N, Yashima M, Huang H L, Fishbein M C, Ting C T, Karagueuzian H S & Chen P S (2001). Pulmonary veins and ligament of marshall as sources of rapid activations in a canine model of sustained atrial fibrillation. Circulation 103, 1157-1163.
Yamada M (2002). The role of muscarinic K(+) channels in the negative chronotropic effect of a muscarinic agonist. J Pharmacol Exp Ther 300, 681-687.
Yamada M, Inanobe A & Kurachi Y (1998). G protein regulation of potassium ion channels. Pharmacol Rev 50, 723-760.
Yue L, Feng J, Gaspo R, Li G R, Wang Z & Nattel S (1997). Ionic remodeling underlying action potential changes in a canine model of atrial fibrillation. Circ Res 81, 512-525.
Yue L, Feng J, Li G R & Nattel S (1996). Transient outward and delayed rectifier currents in canine atrium: properties and role of isolation methods. Am J Physiol 270, H2157-H2168.
Zhang L, Lee J K, John S A, Uozumi N & Kodama I (2003). Mechanosensitivity of GIRK channels is mediated by PKC-dependent channel-PIP2 interaction. J Biol Chem 279, 7037-7047. [Abstract/Free Full Text]
Zhang L M, Wang Z & Nattel S (2002). Effects of sustained beta-adrenergic stimulation on ionic currents of cultured adult guinea pig ventricular myocytes. Am J Physiol Heart Circ Physiol 282, H880-H889.

What is claimed is:

1. A method for identifying a compound for treating atrial fibrillation in a mammal having a heart, said method comprising the steps of:
a. subjecting the mammal to an atrial tachycardia remodeling treatment under conditions leading to a substantial increase in a constitutive Kir3 mediated acetylcholine-dependent potassium current in left atrial cardiomyocytes of the heart of the mammal;
b. isolating an atrial preparation from the heart of the mammal;
c. treating the atrial preparation with the compound;
d. submitting the atrial preparation to stimuli so as to attempt to produce tachyarrythmias;
e. selecting the compound as a likely candidate for the treatment of atrial fibrillation if the compound substantially inhibits tachyarrythmias in the atrial preparation.

2. A method as defined in claim 1, wherein said step of subjecting the mammal to an atrial tachycardia remodeling treatment includes tachypacing the heart of the mammal.

3. A method as defined in claim 1, wherein the compound is selected as a likely candidate for the treatment of atrial fibrillation if the compound substantially inhibits tachyarrythmias in the atrial preparation through an inhibition of a Kir-3 mediated ionic current.

4. A method as defined in claim 3, wherein the compound is selected as a likely candidate for the treatment of atrial fibrillation if the compound substantially inhibits tachyarrythmias in the atrial preparation through an inhibition of a Kir-3 mediated potassium current.

5. A method as defined in claim 4, wherein the compound is selected as a likely candidate for the treatment of atrial fibrillation if the compound substantially inhibits tachyarrythmias in the atrial preparation through an inhibition of a time-dependent Kir-3 mediated, hyper-polarization-activated, potassium current.

6. A method as defined in claim 5, wherein the compound is selected as a likely candidate for the treatment of atrial fibrillation if the compound substantially inhibits tachyarrythmias in the atrial preparation through an inhibition of a constitutive time-dependent Kir-3 mediated, hyper-polarization-activated, potassium current.

7. A method as defined in claim 6, wherein the inhibited current is mediated by at least one of a Kir-3.1 channel and a Kir-3.4 channel.

8. A method for treating a cardiac pathology, said method comprising the administration to a mammal of a therapeutically effective amount of a substance interfering with an acetylcholine-dependent potassium current.

9. A method as defined in claim 8, wherein the mammal is a human.

10. A method as defined in claim 9, wherein the substance inhibits a repolarization current so as to increase a duration of a repolarization phase in cardiomyocytes.

11. A method as defined in claim 10, wherein the cardiac pathology includes atrial fibrillation.

12. A method as defined in claim 10, wherein the repolarization current includes a potassium mediated current.

13. A method as defined in claim 12, wherein the potassium current is acetylcholine-dependent.

14. A method as defined in claim 13, wherein the potassium current is acetylcholine-activated.

15. A method as defined in claim 13, wherein the potassium current is conducted by a Kir3 channel.

16. A method as defined in claim 15, wherein the potassium current is mediated by at least one of a Kir-3.1 channel and a Kir-3.4 channel.

17. A method as defined in claim 12, wherein the potassium current is a constitutive potassium current.

18. A method for treating atrial fibrillation, said method comprising the administration to a mammal of a therapeutically effective amount of a substance inhibiting a constitutive acetylcholine-dependent potassium current.

19. A method as defined in claim 18, wherein the mammal is a human.

20. A constitutive acetylcholine-dependent potassium current embodied in a Kir3 ionic channel.

21. A current as defined in claim 20, wherein the current includes a repolarizing current.

22. A method for identifying a compound for treating atrial fibrillation in a mammal, said method comprising the steps of:
a. providing a cell into which a constitutive Kir3 mediated acetylcholine-dependent potassium current is present;
b. treating the cell with the compound; and
c. selecting the compound as a likely candidate for the treatment of atrial fibrillation if the compound substantially inhibits the constitutive Kir3 mediated acetylcholine-dependent potassium current.
